(19) United States
(12) Patent Application Publication
(10) Pub. No.: US 2024/0358914 A1
(43) Pub. Date: Oct. 31, 2024

(54) APPARATUS FOR IMPROVED MEDICAL INSTRUMENT PUMP CONFIGURATIONS

(71) Applicant: Hydrocision, Inc., North Billerica, MA (US)

(72) Inventors: Alain Tranchemontagne, Warwick, RI (US); Paul Kowalski, Haverhill, MA (US); David J DeRoode, North Billerica, MA (US); Sean Sawdon, North Billerica, MA (US)

(73) Assignee: Hydrocision, Inc., North Billerica, MA (US)

(21) Appl. No.: 18/648,313

(22) Filed: Apr. 26, 2024

Related U.S. Application Data
(60) Provisional application No. 63/462,241, filed on Apr. 26, 2023.

(51) Int. Cl. A61M 5/142; A61M 5/172

(52) U.S. Cl. CPC A61M 5/14236; A61M 5/172

(57) ABSTRACT

Systems and apparatuses for pressurized fluid system handpieces having pumps and pump cartridge mechanisms. The system may have a console, the pump and pump cartridge, and the handpiece, with the pump and pump cartridge configured to deliver high velocity fluid to the handpiece. The pump may comprise the pump cartridge, at least one actuator and a means for driving the at least one actuator. The means for driving the at least one actuator drive fluid from an inlet into the pump cartridge and forces the fluid out of the pump cartridge through an outlet to the handpiece.

### CLAIM OF PRIORITY

This application claims priority from U.S. Provisional Patent Application No. 63/462,241 filed Apr. 26, 2023, the contents of which are incorporated herein by reference.

### FIELD OF INVENTION

The present invention is directed to a pump cartridge system. More specifically, the present invention is directed to a pump cartridge system configured to produce high velocity fluid.

### INTRODUCTION

As technology has progressed, so have the innovations in medical care, which have led to vast improvements in patient care and outcomes over time.

One such medical innovation has been the utilization of pressurized fluids during surgical procedures. Pressurized fluid systems enable surgeons to deliver high pressure fluids as a means for excising diseased tissue amongst other uses. Said fluid systems employ a handpiece capable of exploiting the Venturi effect to simultaneously excise and evacuate material in minimally invasive manners. Moreover, the simultaneous excision and evacuation of debris allows surgeons to reduce the potential harms associated with thermal damage to surrounding tissue as a result of traditional surgical techniques.

Current pressurized fluid system handpieces have transformed the field of medicine by allowing surgeons to perform minimally invasive surgeries. Currently, the pressurized fluid system handpieces are disposable, and employ a pump cartridge, which serves as a sterile barrier to other components of the pressurized fluid system. After one use, the handpiece and pump cartridge are discarded.

While contemporary pressurized fluid system handpieces have drastically improved medical care, they are limited and currently are not cost efficient. Furthermore, components of said handpieces and related pumps are susceptible to degradation and wear resulting from the vast amounts of friction between said components. In addition to the limited run-times and rapid degradation of components, the current pressurized fluid system handpieces are cost prohibitive due to the high prices associated with the disposable pump cartridges being integrated into the handpiece.

Accordingly, it would be desirable to provide cost-efficient pressurized fluid system handpieces. It would be desirable to provide improved pumps and/or pump cartridge mechanisms. Yet further, it would be desirable to increase the cost efficiency of pressurized fluid system handpieces, while simultaneously improving the run-times and reliability of said handpieces.

### SUMMARY

Disclosed herein are apparatuses for improved medical instrument pump configurations. More particularly, apparatuses directed to pumps and pump cartridge mechanisms configured to deliver high velocity fluid to a handpiece.

In one embodiment, the pumps and pump cartridge mechanisms may be integrated or otherwise combined with a pressurized fluid system. The pressurized fluid system may, for example, comprise a console, the pump and pump cartridge, and the handpiece. Of course, other configurations of fluid systems are contemplated. For example, in one embodiment, the pressurized fluid system may further comprise a hose between the pump cartridge and the handpiece.

The pump may comprise a pump cartridge, at least one actuator, and a means for driving the at least one actuator. The actuator may be any actuator that a person of ordinary skill in the art may desire, including, for example, a syringe, a screw, a piston, or a ramming mechanism.

Likewise, the means for driving the at least one actuator may be any means that may be desired, such as mechanical or electrical means. In some embodiments, the mechanical means may provide a reciprocal motion or a rotational motion to the one or more actuators to drive the fluid in the system. In another embodiment, the electrical means may be electrical induction which may provide for movement of the one or more actuators to drive the fluid in the system.

It is contemplated that the invention of the present disclosure may be configured to mitigate overheating and run-time limitations, for example, via robust power delivery solutions. Further, the invention of the present disclosure may prevent rapid wear of components due to friction, for example, by minimizing interfacing wear components. Issues associated with pressure and/or velocity oscillations may be resolved, as described herein, by promoting smooth fluid delivery. Yet further, high costs associated with the disposable pump cartridge (i.e., more than 30% of the device) may be alleviated by supplying a low-cost sterile fluid pathway. Accordingly, the improved pumps and/or pump cartridge mechanisms provided herein may be efficient, intuitive, easily controlled, configured for suction upon intake stroke and infusion upon exhaust stroke, and/or adapted to maintain a fluid ‘vacuum’ once purged (i.e., no air ingress).

As a non-limiting example, therapies may require more power and more reliability than the traditional systems provide. Yet further, an improved pump and/or pump cartridge mechanism may contribute to lowering the cost and waste of a stainless-steel pump cartridge disposable.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 illustrates one embodiment of a pressurized fluid system.

FIG. 2 illustrates a block diagram of one embodiment of a pressurized fluid system.

FIG. 3 illustrates one embodiment of a component of a pump cartridge.

FIG. 4 illustrates one embodiment of a component of a pump cartridge.

FIG. 5 illustrates one embodiment of a pump cartridge.

FIG. 6 illustrates one embodiment of a pump cartridge.

FIG. 7A illustrates one embodiment of a pump cartridge.

FIG. 7B illustrates one embodiment of a pump cartridge.

FIG. 8A illustrates one embodiment of a pump cartridge.

FIG. 8B illustrates the embodiment of the pump cartridge in FIG. 8A.

FIG. 9 illustrates one embodiment of a pump cartridge.

FIG. 10 illustrates one embodiment of the pressurized fluid system.

FIG. 11 illustrates one embodiment of a pump cartridge.

FIG. 12 illustrates an embodiment of a fluid attenuator for use with the pressurized fluid system.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

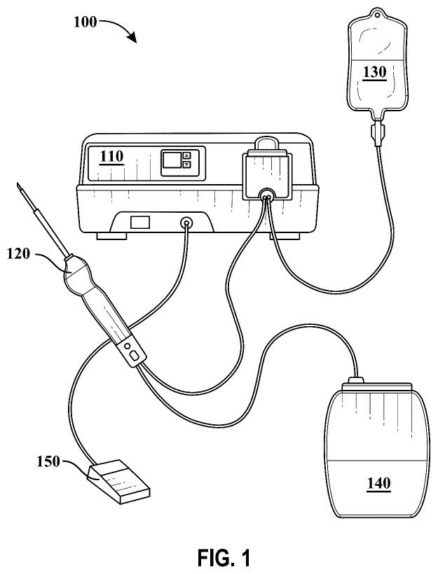

For the purposes of this disclosure, the pumps and pump cartridge mechanisms described herein may be implemented in conjunction, in supplement, and/or as replacements to components of a traditional base system 100 illustrated in FIG. 1.

The base system 100 comprises a console 110, a handpiece 120, a fluid reservoir 130, a waste container 140, and a controller 150. Of course, in some embodiments, the system may be configured in any manner that a person of ordinary skill in the art may desire. Further, in some embodiments, the system may omit or combine any of the aforementioned components according to the needs and desires of one of ordinary skill in the art.

Accordingly, the systems and methods described herein may remove the pump mechanism from handpiece 120 of the base system 100. The base system 100 may be adapted to deliver a high velocity stream of fluid, for example sterile saline, to enable a Venturi suction effect to cut and remove tissue in a minimally invasive manner. In such an embodiment, the high velocity saline cuts and debrides the tissue while simultaneously creating suction to remove material and fluid through the handpiece 120 to the waste container 140. It is contemplated that the pumps and pump cartridges discussed herein may not create thermal damage to surrounding tissue as tissue can be sub-selectively cut by adjusting the system pressure. The base system 100 may be configurable with multiple styles of handpiece 120 for a variety of therapies, including tenotomy, discectomy and fusion, and allografts.

The base system 100 may comprise a pump cartridge integrated into a disposable. For example, the disposable may be configured as the handpiece 120 and may comprise the pump cartridge. This traditional pump cartridge may be a single stroke piston type mechanism that consists of precision machined stainless steel and a complex geometry of valves and seals. In an embodiment, this cartridge may be used on each disposable to provide the interface to the console and may serve as the sterile barrier to the console. This disposable, including the pump cartridge, may be disposed after every use.

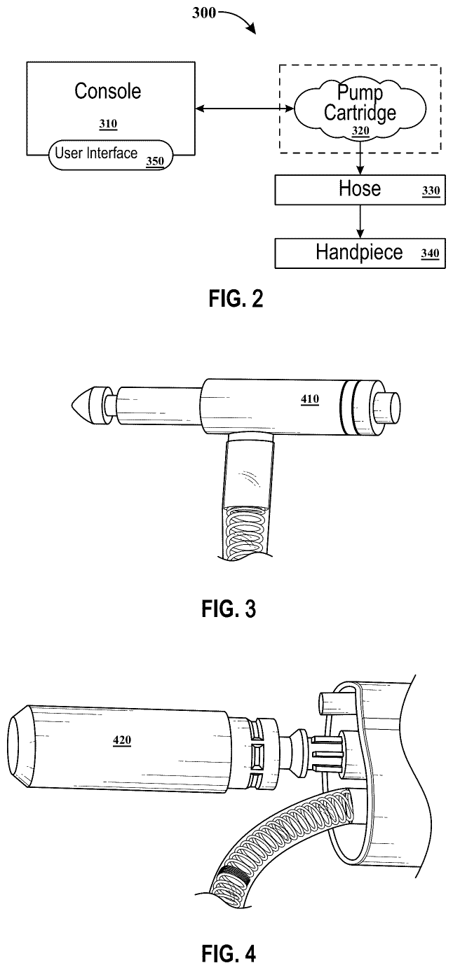

FIG. 2 illustrates a block diagram of one embodiment of a high-pressure system 300 according to the present disclosure. The system 300 comprises a console 310, a pump cartridge 320, a hose 330, and a handpiece 340. Reference is made throughout to use with a handpiece 340, however, a catheter or other attachment may be utilized. In one embodiment, the console 310 may comprise a user interface 350. The user interface 350 may be any user interface that a person of ordinary skill in the art may desire. For example, the user interface 350 may be configured as a foot pedal, a graphical user interface, buttons, or any other interface that may be desired.

Returning to the embodiment in FIG. 2, the pump cartridge 320 may comprise a pump input end in conjunction with the console 310 and a pump output end in conjunction with the hose 330 and handpiece 340. It is contemplated that in some embodiments additional components may be disposed positioned in the system.

The pump may comprise a pump cartridge 320 mechanism, at least one actuator, and a means for driving the at least one actuator. For the purposes of this disclosure, the pump and/or pump cartridge 320 mechanism may be configured to deliver high velocity fluid to the handpiece 340 to facilitate a broad concept generation workspace. Thus, the end-use or handpiece 340 compatibility of the pump and/or pump cartridge 320 mechanism should not be deemed as limiting.

Any of the pump and pump cartridge 320 mechanism may be compatible with the hose 330 configured as a high-pressure supply line to allow operation from a non-sterile area to the sterile area of an operating suite. For example, in one embodiment, the high-pressure supply line may be about 10 feet in length. However, the pump may be compatible with a high-pressure supply line of any suitable length. Further, in one embodiment, any of the pump and pump cartridge 320 mechanism may be compatible with a pump outlet pressure of approximately 15,000 psi and a flow rate of approximately 225 ml/min. However, in other embodiments, the pump may be compatible with any pump outlet pressure. In one embodiment, the pump and/or pump cartridge 320 mechanism may be compatible with an approximately 5-foot-long catheter, for example, operating as the handpiece 340 in a vascular application. Yet further, the systems described herein may be adapted to prevent over-heating and/or undesired shut down, for example, configured for runtimes varying from 5 to 60 minutes of continual use with potential back-to-back use with nonburdensome downtime between. In a further embodiment, the system may be configured for runtimes as short as a few seconds and as long as 90 minutes. However, the system may be configured for any suitable runtimes.

Although each embodiment of the pumps and pump cartridge 320 mechanisms described herein may include embodiment-specific characteristics, there exist functional aspects that span more than one embodiment. For example, in one embodiment, the system 300 may utilize at least one actuator configured as a reciprocating single stroke piston that is integrated into the handpiece 340. In such an embodiment, the at least one actuator draws in fluid on a backstroke and pressurizes the system on a forward stroke by use of one-way valving. Further, in such an embodiment, the system 300 incorporates a unique coupling system to the console 310 that latches to the at least one actuator and can identify the handpiece 340 to configure a therapy mode within the console. In an embodiment, the console 310 provides the power to reciprocate the piston within the cartridge body. FIGS. 3 and 4 illustrate an embodiment of a disassembled pump cartridge piston 410 and body 420, respectively.

In an embodiment, the base system piston action may comprise reciprocating motion within an actuator body, creating a high-pressure output of a fluid drawn into the pump cartridge from a fluid reservoir. In one embodiment, the fluid reservoir may be an attached unpressurized saline bag. However, any fluid reservoir that a person of ordinary skill in the art may desire is contemplated.

In various embodiments, the outlet pressure is specified as a maximum of 15,000 psi. However, other suitable maximum outlet pressures are possible. A person of ordinary skill in the art will recognize that based on the outlet pressure, current geometry of the handpiece, and measured flow rate, other critical calculations can be estimated such as fluid velocity, head pressure, and the work done by the system. In an embodiment, pumps and pump cartridge 320 mechanisms are configured to perform the “work” to accelerate the fluid as close to the patient as possible and maintain as large of tubing diameters as possible throughout the system. In one embodiment, any of the pump or pump cartridge 320 may comprise a chamfer between any of the hose or handpiece. It is contemplated that the chamfer may reduce pressure losses between the components. Therefore, the pumps and pump cartridge mechanisms may be configured to provide a high velocity fluid and reduce the cost/complexity/waste of the base system disposable.

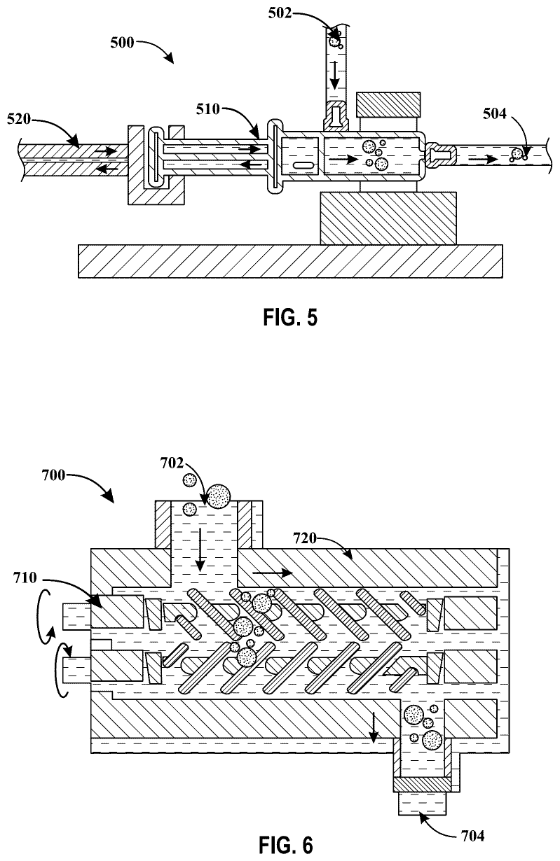

Referring to FIG. 5, one embodiment of the pump cartridge mechanism, referred to as a pump cartridge syringe 500, is illustrated. The pump cartridge syringe 500 comprises an inlet 502 and an outlet 504, at least one actuator configured as a syringe 510, and a housing 512. As illustrated, the syringe 510 may be configured as a plunger, however, any syringe that a person of ordinary skill in the art may desire is contemplated. Returning to the embodiment of FIG. 5, the syringe 510 may be associated with the housing 420 and operative to drive fluid in the system. From a functionality perspective, the pump cartridge syringe 500 may operate by pulling back on the syringe 510 to draw fluid into the housing 512, then pushing to force the fluid out at high pressure. In various embodiments, the geometry of the syringe 510 stroke and bore may be sized to create the preferred downstream velocity. In a further embodiment, the geometry of the pump cartridge may comprise a chamfer contemplated to reduce pressure losses at an outlet of the pump cartridge, thus reducing pressure loss on the high pressure fluid.

The pump cartridge syringe 500 may be retrofitted and/or may utilize the method of action of a preexisting console. In an embodiment, the stroke and bore of the pump cartridge syringe 500 embodiment may be greater with slower rotational speed (RPM), generating a greater pressure gradient. A sleeve or clamshell may be included within the pump cartridge syringe 500 embodiment, wherein the sleeve or clamshell may be configured to contain pressure. In one embodiment, the sleeve or clamshell may be a metal sleeve or clamshell. However, any suitable material may be utilized and the aforementioned are provided as non-limiting examples. One or more components of the pump cartridge syringe 500 may be interchangeable, for example, manifesting as a modular cartridge. Accordingly, in some embodiments, the fluid path may be separated, for example, to maintain sanitary conditions between two or more fluids and/or two or more pump cartridge components. However, in another embodiment the pump cartridge syringe 500 may be a unitary cartridge.

In one embodiment, the pump cartridge syringe 500 may be configured to receive a sealed container, wherein said sealed container may keep injected fluid sterile and, importantly, may create pressurization. In an embodiment, the sealed container may be a saline pouch, isolating sterile fluid from a reusable environment. In such an embodiment, the scaled container may be flexible. In some embodiments, the scaled container may be capable of accepting pressure, allowing pressurization and propulsion of the contained fluid, while maintaining separation and sterility with the overall pump cartridge device. Therefore, the pump cartridge syringe 500 allows a container to be placed within the pump cartridge such that the container does not rupture. While reference is made to an embodiment wherein the container is the bag of saline, any suitable container may be utilized.

In an embodiment, the pump cartridge syringe 500 may utilize the syringe 510 in conjunction with the pump of the base system, wherein the pump is a means for driving fluid in the system. The syringe 510 may comprise any material that may be desired, including, for example low-cost polycarbonate, composite, or similar plastic.

The syringe 510 may be placed inside the housing 512 configured to shed the radial stress load from the pressure created. As a non-limiting example, the syringe 510 serves as a sterile barrier inside an existing pump. This housing may then shift the loading on the syringe from a radial tensile stress of a pressure vessel to a compressive “crush” force, which most rigid plastics can handle. In one embodiment, the housing 512 may be a metal housing, however, any material may be utilized.

It is contemplated that the embodiment as shown in FIG. 5 may allow for handpieces and/or catheters to be swapped during a procedure without resetting the console. Further, in such an embodiment, if an unforeseen event were to occur at the console side, a catheter and/or handpiece may be disconnected, and procedural placement can be maintained while a new cartridge is setup.

In an embodiment, a portion of the pump cartridge mechanism below may be disposable. In various embodiments, a durable piston or ramrod portion may be exposed to an antiseptic (i.e., ChloraPrep) and/or otherwise sterilized; and/or a replaceable seal may be integrated to maintain a fluid barrier.

Accordingly, the pump cartridge as shown in FIG. 5 may integrate the syringe 510 into the console itself, creating a sturdy and durable component. For example, the syringe 510 may comprise any actuator known in the art, including, for example and without limitation, a plunger or a piston. Thus, the configuration as shown in FIG. 5 may reduce the disposable cost, which may be significant depending on the size requirements of the syringe 510.

In some embodiments, the means for driving the actuator 520 may be a mechanical means. For example, the console, or other means, may affect mechanical movement, such as reciprocal motion, on the actuator to drive fluid within the system.

Referring to FIG. 6, an embodiment of a pump cartridge 700 mechanism comprising a variable pitch screw 710, a housing 720, an inlet 702, and an outlet 704 is illustrated. Such a variable pitch screw 710 may function similar to an extruder mechanism, wherein changing pitch compresses and/or accelerates fluid contained within the housing 720. In one embodiment, the variable pitch screw 710 and/or components thereof may be composed of injection molded parts. In a further embodiment, the variable pitch screw 710 may be configurable in forward and reverse, for example, to instigate differed modes of aspiration. In various embodiments, the pump cartridge mechanism may include any suitable number of variable pitch screws 710, for example, one-screw, two-screws, or even more screws.

In an embodiment, the variable pitch screw 710 is enclosed within a housing 720 and delivers fluid to the outlet 704. For example, in one embodiment, the variable pitch screw 710 may be encased in a cylindrical housing 720. Of course, other housing 720 configurations that a person of ordinary skill may desire are contemplated. In yet a further embodiment, fluid is able to move into the system by exploiting gravitational forces. Moreover, multiple variable pitch screws 710 may be utilized to increase fluid acceleration.

Of course, other embodiments of the pump cartridge mechanism may utilize a variety of screws. In one embodiment, the pump cartridge mechanism may comprise an Archimedes screw. In another embodiment, the pump cartridge mechanism may comprise a lead screw. In yet a further embodiment, the pump cartridge mechanism may comprise two screws. In still another embodiment, the pump cartridge mechanism comprises a translational screw.

In another embodiment, an ancillary peristaltic pump is used to move fluid into the system. It is contemplated that any peristaltic pump that a person of ordinary skill in the art may desire may be utilized. For example, the peristaltic pump may be any of a trilobe peristaltic pump, a many-lobe peristaltic pump, a peristaltic pump with an in-series amplifier, an electromagnetic multistage peristaltic pump, and a finger trap peristaltic pump.

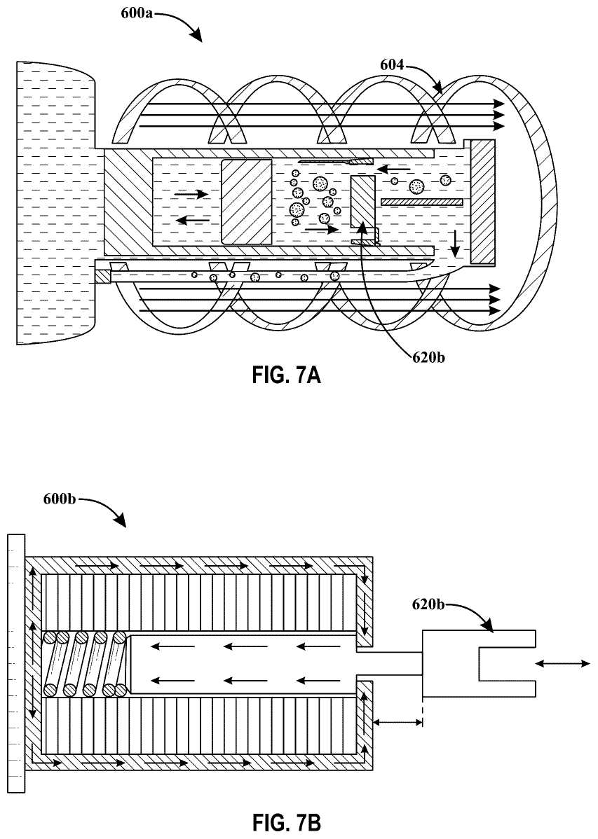

Referring to FIGS. 7A-7B, an embodiment of a pump cartridge mechanism 600a,b utilizing magnetic induction to drive an actuator, for example, at high power and/or speed is illustrated. In the embodiment illustrated, the actuator may be a piston 620a,b. Such an embodiment may reduce the number of required moving parts. Thus, such an embodiment may provide reliable cycling and functionality.

As shown in FIGS. 7A-7B, magnetic induction may be utilized to reciprocate the piston. Such an embodiment may reduce the electrical work and vibration of the mechanical interfaces used to operate the cartridge. In another embodiment, not shown, the pump cartridge syringe 500 shown in FIG. 5 may be retrofitted with a ferromagnetic material. For example, in one embodiment, the syringe 510 may comprise a plug comprising the ferromagnetic material. In such an embodiment, magnetic induction 604, illustrated in FIG. 7A, may drive the actuator, such as the plug or piston 620a, back and forth in reciprocating motion within the syringe.

In another embodiment, a bank of smaller actuators working like a multi-piston “engine” may be configured with a valved manifold to provide adequate power.

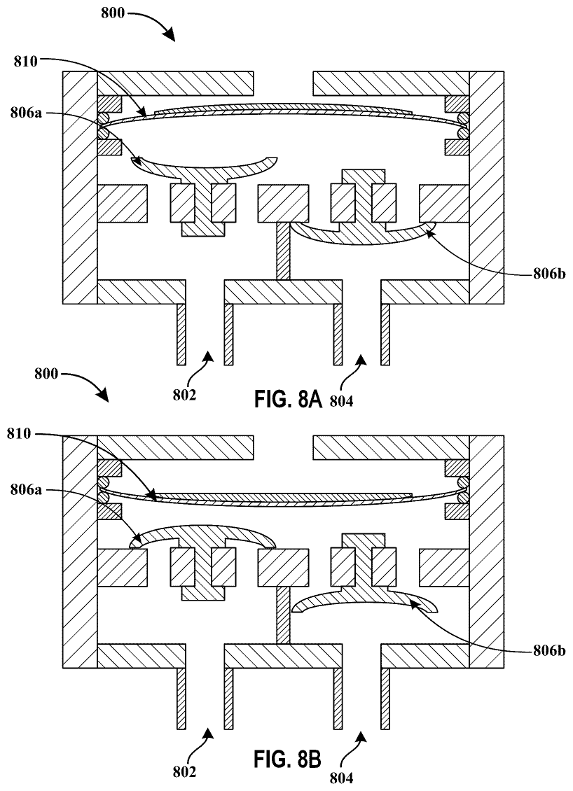

FIGS. 8A and 8B illustrate an embodiment of a pump cartridge 800 comprising an actuator for moving fluid, an inlet 802, and outlet 804, and at least one valve 806a,b. In one embodiment, the pump cartridge may comprise a transducer 810 configured as the actuator capable of converting electrical energy into mechanical displacement. In an embodiment, the transducer 810 is a piezoelectric actuator. However, the transducer 810 encompassing the conversion of electrical energy into mechanical displacement may include any suitable transducer alternative.

Returning to the embodiments illustrated in FIGS. 8A and 8B, the transducer 810 may oscillate in a linear fashion from the state shown in FIG. 8A to the state shown in FIG. 8B. As illustrated, the oscillation of the transducer 810 may result in the at least one valve 806a,b opening and closing to control fluid flow. In an embodiment, the piezoelectric actuator oscillates in excess of one million cycles per second moving fluid through microchannels. However, in another embodiment, the transducer 810 may oscillate between 100,000 and 300,000 cycles per second. In various embodiments, the transducer may oscillate below 100,000 cycles per second, above 300,000 cycles per second, or at any suitable rate.

In some embodiments, the system may heat the fluid during use. In an embodiment, the system may boil the fluid through energy transfer at a molecular level to move the fluid through microchannels to create sufficient velocity to move the fluid through the system. Boiling the fluid may result in the fluid being in a gaseous state, such as a gas or vapor. Thus, in such an embodiment, gas may be utilized at the pumping phase, wherein the gas may condense before being delivered to the patient. Further, in some embodiments, the gas may be utilized at a pressurization phase.

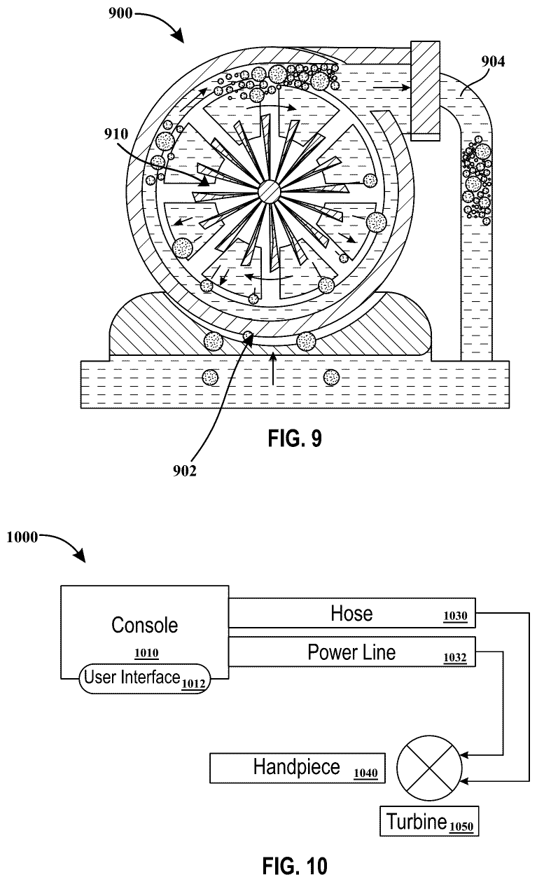

FIG. 9 illustrates an embodiment of a pump cartridge 900 utilizing rotational motion for moving a fluid. The pump cartridge 900 may comprise an actuator configured as a rotational motion mechanism 910 to move fluid from an inlet 902 to an outlet 904. In an embodiment, the rotational motion mechanism 910 is an impeller. In a further embodiment, the rotational motion mechanism 910 is a turbine.

As a non-limiting example, a means for driving the at least one actuator comprises energy used to spin a drive component on the console side of the system, which is attached to a corresponding disposable impeller that will draw in fluid from the inlet 902 and rapidly accelerate it through the outlet 904 and a disposable tubing and/or sub-system secured to the outlet 904. The rotational motion mechanism 910 may be sealably attached to the console, wherein said attachment may be achieved by magnetic coupling. However, in other embodiments, the attachment may be achieved by any means that a person of ordinary skill in the art may desire. Furthermore, in one embodiment the rotational motion mechanism 910 may be disposable. In some embodiments, the pump cartridge 900 may utilize more than one rotational motion mechanism as a means for driving fluid. It is contemplated that the means for driving the at least one actuator may increase the velocity of which fluid moves.

Additionally, the rotational motion mechanism 910 may be actuated by the means for driving the at least one actuator. In an embodiment, the means for driving the at least one actuator is electrically controlled. In another embodiment, the means for driving the at least one actuator is controlled by air. In yet a further embodiment, the means for driving the at least one actuator is operated by utilizing fluid power.

In one embodiment, the rotational motion mechanism 910 may comprise an acceleration means configured to accelerate the fluid. In some embodiments, the rotational motion mechanism 910 may be a paddle wheel, wherein each paddle may push any of the fluid. In one embodiment, the rotational motion mechanism 910 may be a centrifugal mechanism with an orifice configured to push the fluid. In a further embodiment, the rotational motion mechanism may be configured as a particle accelerator configured to drive the fluid.

In an embodiment, the pump cartridge may comprise a flywheel as a means for moving fluid. In an embodiment, the flywheel is capable of storing fluid, wherein said fluid is spun within a container that stores energy in the form of momentum and/or inertia. In another embodiment, the electrical energy is built up over time, utilizing a mechanical flywheel, wherein the mechanical flywheel engages with the system to spin other components at high speed and/or torque.

In an embodiment, illustrated in FIG. 10, a system 1000 may comprise a console 1010, a hose 1030, a power line 1032, a handpiece 1040, and a pump configured as at least one turbine 1050 operative to increase fluid velocity in the system. In one embodiment, a plurality of the at least one turbines 1050 may be placed in series or “stages” to create an additive increase in velocity. Such an embodiment may include iterative development of gearing and staging. Such a system 1000 may provide the working energy at the point of use as energy that can be transmitted to the handpiece 1040 through the power line 1032 to actuate the turbine 1050 remotely. The power line 1032 may be any of an electrical, air, or fluid line power line. The hose 1030 may be any hose that a person of ordinary skill in the art may desire, including, for example and without limitation, a low pressure hose.

In an embodiment, any of the mechanical linkage may be divorced from the console 1010. In such an embodiment, the console 1010 may comprise a user interface 1012 outside the sterile field where fluid and the handpiece energy is then transmitted to a working handpiece 1040 unit at the patient. In one embodiment, the fluid may be a low pressure sterile saline, however, any fluid that a person of ordinary skill may desire is contemplated. Further, it is contemplated that this design may eliminate the need to overcome the pressure loss in traditional high-pressure hoses.

In one embodiment, the turbine 1050 may be configured as a pump. However, in another embodiment, not illustrated, the system may further comprise a pump. In such an embodiment, the pump may be disposed along the hose and/or power line between the console and the turbine, between the turbine and the handpiece, or even within the handpiece. However, the aforementioned configurations are provided as non-limiting examples only and should not be considered limiting.

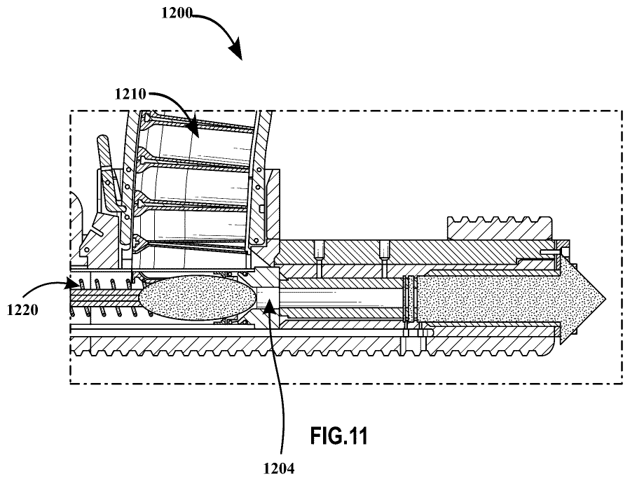

FIG. 11 illustrates an embodiment of a pump cartridge 1200, wherein the pump cartridge 1200 utilizes pelletized fluid to aid in the movement of fluids. In an embodiment, the pelletized fluid is configured as consumable water packets 1210 fed individually into the system. The consumable water packets 1210 are configured in any way that may be desired, for example, as pellets, beads, or other water packets. In another embodiment, an actuator configured as a high velocity ramming mechanism 1220 crushes the consumable water packets 1210 against a pressure outlet 1204 to force the consumable water packet 1210 to break and for the fluid to be accelerated into the outlet 1204. In yet a further embodiment, the empty consumable water packet 1210 is actuated out of the system, wherein the actuation may occur at exceedingly high speeds to ensure the continuous flow of fluid.

While the aforementioned embodiments describe a single actuator, it is contemplated that any number of actuators may be utilized. For example, in one embodiment, the actuator may be two or more actuators positioned in series. It is contemplated that positioning pistons in series may reduce forces within the system.

In another embodiment, the actuators may be two or more actuators in parallel, for example, in a crank or CAM shaft configuration or V2 engine configuration. It is contemplated that the actuators being placed in parallel may reduce oscillations in the system, for example, at the tip of the handpiece.

In still another embodiment, the actuator may be a single actuator configured to move between a plurality of chambers. For example, a dual path reciprocating piston, such as a mechanical heart, may be utilized.

In some embodiments, the pump cartridge may comprise a flexible container to drive the fluid. For example, the flexible container may be a flexible membrane. In one embodiment, the flexible container may drive the fluid through the application of a non-sterile oscillating motion which compresses the flexible container. The flexible container may be compressed by an actuator, such as a piston, which directly contacts the flexible membrane. However, in another embodiment, the flexible container may be compressed by hydraulic fluid. In such an embodiment, the pump cartridge may comprise a permanent membrane in contact with the flexible membrane that applies a force to the flexible membrane, driving fluid into the system.

In an embodiment, the pump, pump cartridge, or other component of the system, may comprise cleaning mechanisms. In one embodiment, the cleaning mechanisms may utilize physical sterilization such as heat, radiation, or filtration. For example, physical sterilization comprising heat may include heating the component of the system to a temperature known to kill contaminants through dry or moist means. In another embodiment, the cleaning mechanism may utilize chemical sterilization such as gas or liquid sterilization. For example, the chemical sterilization may comprise a gaseous sterilization, such as formaldehyde or ethylene oxide, or a liquid sterilization, such as alcohol, halogens, phenols, or aldehydes. A person of ordinary skill in the art will recognize that the aforementioned sterilization mechanisms are provided as examples only and any sterilization mechanism may be utilized.

In an embodiment, a purge step may be implemented at the start of the procedure that would utilize chemical sterilization to sterilize the system. In one embodiment, the purge step may comprise pumping a chemical sterilization solution through the entire system to flush the system prior to use. The chemical sterilization solution may be any solution that a person of ordinary skill in the art may desire, including, for example and without limitation, any of alcohol, chlorine, and peroxide type solutions. In an embodiment, a dye can be implemented to ensure visual cues are easily understood by the end user during sterilization. Accordingly, this stage may ensure any durable component that may have trace and/or residual contamination is sufficiently sterilized.

Similarly, embodiments utilizing metal components to house other disposable pieces may comprise heating elements. As a non-limiting example, between cases, these heating elements could bake out any residual moisture and kill off microbial contamination that may be present within interfacing components. In various embodiments, other sterilization mechanisms may be utilized, such as steam, UV light, and ethylene oxide dosing.

In an embodiment, the pump cartridge may utilize a venturi effect to bring fluid into the system. It is contemplated that the venturi effect may provide a lack of complexity, such as electrical or mechanical complexity, to pull fluid into the system. In some embodiment, a similar venturi effect may occur within the handpiece.

In an embodiment, non-pressurized fluid and pressurized air may be routed to the patient, where the acceleration of the fluid through the venturi effect can happen directly at the point of use. For example, such a concept may utilize the functionality of the impeller/turbine of FIG. 9. Such a concept may limit the mechanical complexity, as venturi tubes may be configured without moving parts.

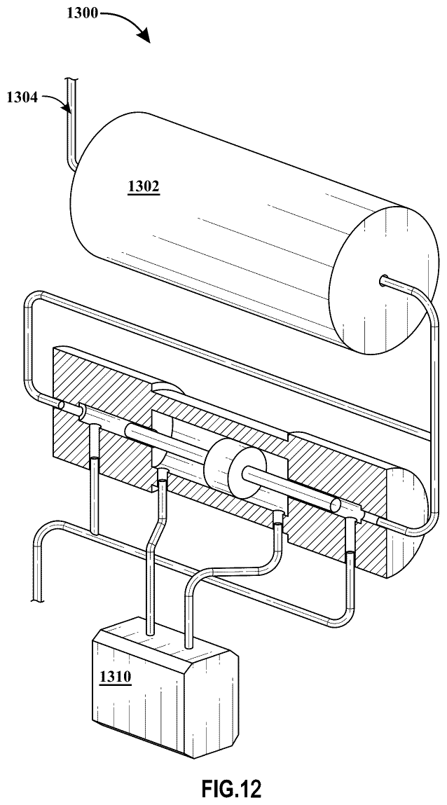

In some embodiments, the system may utilize pressure wave attenuation. In an embodiment illustrated in FIG. 12, a fluid attenuator 1300, comprising a tank 1302 is disposed prior to a fluid outlet 1304 to dampen any pulsatile waves generated with the pump 1310. Once the system is fully purged and ready for operation, the pressurized fluid may pass into the tank 1302 where the vibrations may be dampened by the bolus of fluid contained within. However, as the fluid is incompressible, for every unit of volume entering the tank 1302, an equivalent amount must leave. Therefore, a constant pressure and flow may be maintained with the benefit of removing the vibrations generated by the pump 1310. It may be preferable to reduce pulsatile vibrations and to incorporate vibrational solutions into the base system and/or the other pump embodiments described above by means of an addition to the high-pressure tubing near the pump 1310 and pump cartridge.

Accordingly, the attenuator and related components may be incorporated into any one of the pump embodiments described above.

The pump configurations disclosed herein offer versatile applications across various medical devices. These pump designs can be seamlessly integrated into any portion of a medical device, providing a reliable mechanism for the management and delivery of pressurized fluid or gas. Furthermore, the flexibility of these configurations allows for their utilization in combination with other components within a medical device, thereby enhancing the device's functionality and adaptability. Moreover, the disclosed pump designs can be easily modified to suit the specific requirements and objectives of different medical devices while remaining consistent with the fundamental principles underlying the respective medical device. Thus, these pump configurations find utility in a wide range of medical devices and tools, whether facilitating the controlled expulsion of fluid or gas through the patient-end of a tool or enabling the precise actuation of desired motions or effects within the patient-end of various medical instruments, wherein said motions or effects are fluid or gas facilitated. The pumps and pump cartridge designs described herein may be utilized in handheld devices, medical device consoles, or within any suitable subcomponent of a medical device system.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A pressurized fluid system comprising:
a console;
a handpiece; and
a pump comprising a pump cartridge mechanism, at least one actuator, and a means for driving the at least one actuator, wherein the pump comprises an inlet in communication with the console and an outlet in communication with the handpiece.

2. The pressurized fluid system of claim 1, wherein the pump cartridge is configured as a syringe comprising a syringe body and a plunger, wherein pulling back on the plunger draws fluid into the syringe body and pushing the plunger forces the fluid out of the outlet at high pressure.

3. The pressurized fluid system of claim 1, wherein the actuator is configured as at least one screw configured to drive fluid in the system.

4. The pressurized fluid system of claim 1, wherein the actuator is configured as a rotational motion mechanism.

5. The pressurized fluid system of claim 1, wherein the pump cartridge utilizes pelletized fluid to aid in movement of fluid.

6. The pressurized fluid system of claim 1, wherein the pump cartridge utilizes a venturi effect to draw fluid into the system.

7. The pressurized fluid system of claim 1, wherein the means for driving the at least one actuator is a mechanical means.

8. The pressurized fluid system of claim 1, wherein the means for driving the at least one actuator is an electrical means.

9. A pressurized fluid system comprising:
a console comprising a user interface;
a handpiece;
a hose;
a pump cartridge mechanism comprising an inlet in communication with the console and an outlet in communication with the handpiece via the hose;
at least one actuator; and
a means for driving the at least one actuator,
wherein the pump cartridge serves a sterile barrier within the system.

10. The pressurized fluid system of claim 9, wherein the pump cartridge is configured as a syringe comprising a syringe body and a plunger, wherein pulling back on the plunger draws fluid into the syringe body and pushing the plunger forces the fluid out of the outlet at high pressure.

11. The pressurized fluid system of claim 9, wherein the actuator is configured as at least one screw configured to drive fluid in the system.

12. The pressurized fluid system of claim 9, wherein the actuator is configured as a rotational motion mechanism.

13. The pressurized fluid system of claim 9, wherein the pump cartridge utilizes pelletized fluid to aid in movement of fluid.

14. The pressurized fluid system of claim 9, wherein the pump cartridge utilizes a venturi effect to draw fluid into the system.

15. The pressurized fluid system of claim 9, wherein the means for driving the at least one actuator is a mechanical means.

16. The pressurized fluid system of claim 9, wherein the means for driving the at least one actuator is an electrical means.

17. The pressurized fluid system of claim 9, wherein the hose is a high-pressure supply line and the pump cartridge is compatible with a pump outlet pressure of about 15,000 psi and a flow rate of approximately 225 ml/min.
